(19) United States
(12) Patent Application Publication
(10) Pub. No.: US 2007/0118131 A1
(43) Pub. Date: May 24, 2007

(54) Anchor for Augmentation of Screw Purchase and Improvement of Screw Safety in Pedicle Screw Fixation and Bone Fracture Fixation Systems

(76) Inventor: Hubert L. Gooch, Concord, NC (US)

Correspondence Address: Kevin Alan Wolff, 209 Providence Road, Chapel Hill, NC, 27514, US

(21) Appl. No.: 11/550,402

(22) Filed: Oct. 17, 2006

Related U.S. Application Data
(60) Provisional application No. 60/596,734, filed on Oct. 17, 2005.

(51) Int. Cl. A61B 17/58

(52) U.S. Cl. 606/72

(57) ABSTRACT

The present invention is directed generally to a biomedical device(s). More specifically, some embodiments may include a device(s) used in orthopedic treatment(s). Some embodiments may include, for example, a device(s) for improvement of spine pedicle screw fixation and safety. Further, some embodiments may also include, for example, a device(s) that may be utilized in orthopedic fracture fixation. Still some embodiments may further include, for example, a device(s) providing improved screw fixation and enhanced stabilization in fracture repair. Some embodiments may further include, for example, a device(s) that expands upon insertion of a pedicle screw or cortical screw into the appropriate device(s).

This application claims the benefit of U.S. Provisional Application No. 60/596,734 to Hubert Gooch entitled ANCHOR FOR AUGMENTATION OF SCREW PURCHASE AND IMPROVEMENT OF SCREW SAFETY IN PEDICLE SCREW FIXATION AND BONE FRACTURE FIXATION SYSTEMS filed Oct. 17, 2005, the entire disclosure of which is hereby incorporated by reference as if set forth fully herein.

This disclosure may contain information subject to copyright protection. The copyright owner has no objection to the facsimile reproduction by anyone of the patent disclosure or the patent as it appears in the U.S. Patent and Trademark Office files or records, but otherwise reserves all copyright rights whatsoever.

### BACKGROUND

### Field of the Invention

The present invention relates to the field of biomedical devices, more specifically, a device(s) used in orthopedic treatment(s).

### SUMMARY

The present invention is directed generally to a biomedical device(s). More specifically, some embodiments may include a device(s) used in orthopedic treatment(s).

Some embodiments may include, for example, a device(s) for improvement of spine pedicle screw fixation and safety. Further, some embodiments may also include, for example, a device(s) that may be utilized in orthopedic fracture fixation. Still some embodiments may further include, for example, a device(s) providing improved screw fixation and enhanced stabilization in fracture repair. Some embodiments may further include, for example, a device(s) that expands upon insertion of a pedicle screw or cortical screw into the appropriate device(s).

Still further aspects included for various embodiments will be apparent to one skilled in the art based on the study of the following disclosure and the accompanying drawings thereto.

### BRIEF DESCRIPTION OF THE DRAWINGS

The utility, objects, features and advantages of the invention will be readily appreciated and understood from consideration of the following detailed description of the embodiments of this invention, when taken with the accompanying drawings, in which same numbered elements are identical and:

FIG. 1 is an exemplary embodiment of an anchor, according to at least one embodiment;

FIG. 2 is an exemplary embodiment of an anchor, according to at least one embodiment;

FIG. 3 is an exemplary embodiment of an anchor, according to at least one embodiment;

FIG. 4 is an exemplary axial view vertebral body depicting a pedicle screw, a pedicle channel, and an expanded bone/pedicle anchor, according to at least one embodiment;

FIG. 5 is an exemplary lateral view vertebral body depicting a pedicle channel, a pedicle screw, and an expanded bone/pedicle anchor, according to at least one embodiment;

FIG. 6 is an exemplary superior view vertebral body depicting a pedicle channel, a pedicle anchor, and a passage for expansion screw or pedicle screw, according to at least one embodiment.

### DETAILED DESCRIPTION

Spinal instrumentation with pedicle screws is a common and daily occurrence in the treatments of various spine pathologies. Several factors can frequently make spinal instrumentation or any form of bone instrumentation more difficult. An example of a patient population frequently found to require spine surgery is the elderly. Surgical stabilization in this population of patients is especially challenging because of the frequent finding of osteoporosis, which makes the screw to bone interface of routine spinal instrumentation techniques more precarious.

Increased screw pull-out, loosening and fracture of instrumented pedicles is unfortunately a problem in this patient demographic. Reconstructive spine surgery for failed fixation often encounters situations where the previously instrumented pedicle canal has been expanded by toggling and loosening of the screw. This makes it difficult to implant another screw at this site making the fixation more tenuous across the level in question.

During the process of establishing a pedicle channel for placement of a pedicle screw, the pedicle cortical wall can inadvertently be broached. If the cortical broach is situated inferiorly or medially, there is potential risk of damage or irritation to the spinal cord or nerve roots. In the realm of general orthopedic surgery, bone quality and the ability to gain adequate screw purchase is critical in fracture fixation.

Osteoporotic bone makes screw purchase with cortical screws difficult, if not impossible, during the course of fracture fixation. The present invention proposes a novel solution to these various problems by utilizing a device to enhance the screw to bone interface and strengthen screw pullout, thereby improving spinal or fracture fixation.

In at least one embodiment of the present invention, the bone anchor device is comprised of, for example, allograft, PEEK, carbon fiber, or other biomaterial.

In at least one embodiment of the present invention, the walls would be threaded externally to allow the present invention to be threaded into a previously tapped pedicle or long bone.

At least one embodiment of the present invention would be manufactured in such a way as to allow expansion of the walls of the present invention with the insertion of a pedicle screw or cortical screw.

In at least one embodiment, placement of the present invention would cover any broaching of the pedicle canal and protect the exposed nerve root or spinal cord.

In at least one embodiment of the present invention, expansion of the walls of the present invention would result in lateral forces expanding outward against the pedicle walls. In addition to pullout strength afforded by the threads of the present invention interdigitating with the threads of the pedicle channel, the lateral forces would improve pullout strength within the pedicle as well as a portion within the vertebral body.

The present invention may be utilized, for example, in fracture surgery in osteoporotic bone would allow improved cortical fixation by means of lateral wall expansion within the weakened cortical bone. The present invention may be manufactured with an expansive tip that would expand with screw insertion resulting in increased pullout strength by filling the cortical hole on the far side of the cortical bone. In this manner, screw purchase would not depend on cortical screw fixation with threads alone, but rather would be augmented by the lateral forces applied by expansion of the present invention within the cortices as well as the expanded tip of the present invention.

The present invention may be utilized in, for example, revision spine surgery. At least one embodiment of the present invention would be manufactured in various sizes that would be large enough to fill any large pedicle expansion resulting from toggling and loosening of a previously placed pedicle screw.

At least one embodiment of the present invention would provide a transition between the pedicle screw fixation constructs and the underlying bone. This transition effect would potentially prevent or minimize toggling and screw loosening resulting from micro motion of the screw.

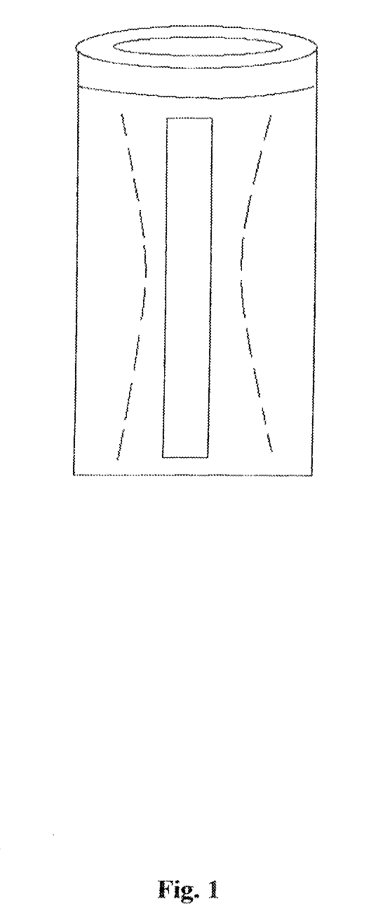

Referring to FIG. 1, an embodiment of an anchor is shown.

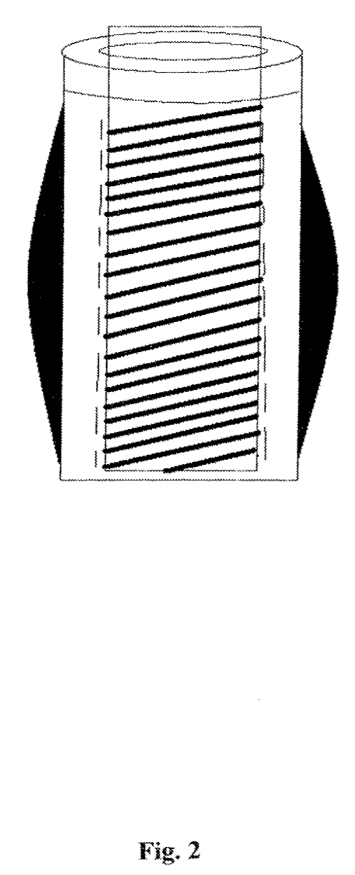

Referring to FIG. 2, an embodiment of an anchor is shown.

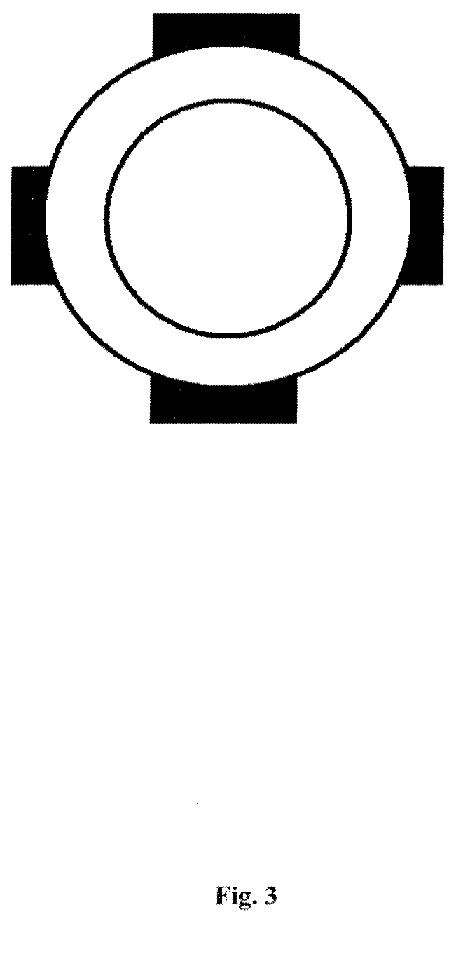

Referring to FIG. 3, an embodiment of an anchor is shown.

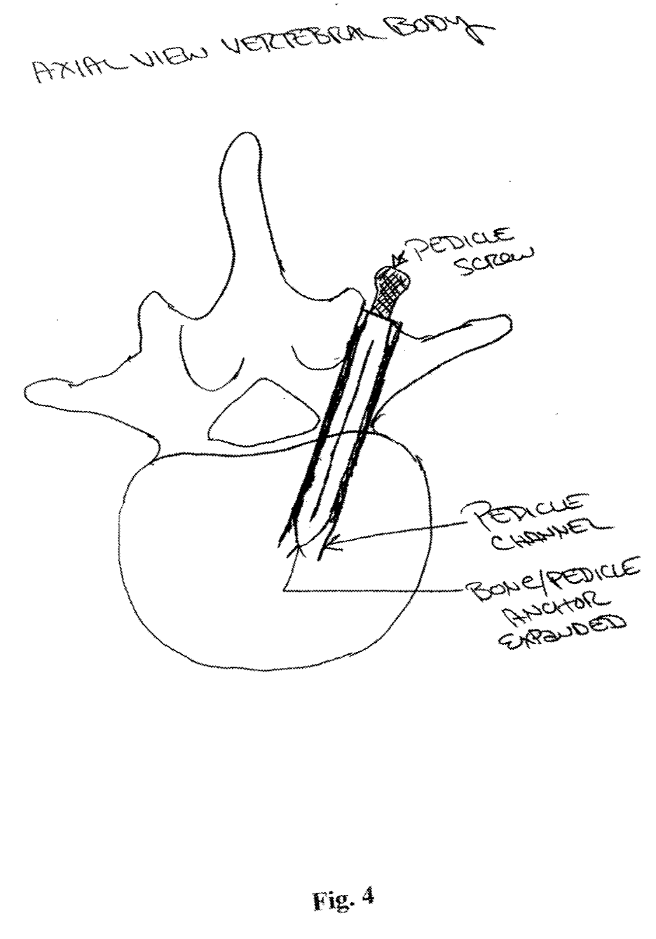

Referring to FIG. 4, an axial view vertebral body is shown which depicts a pedicle screw, a pedicle channel, and an expanded bone/pedicle anchor.

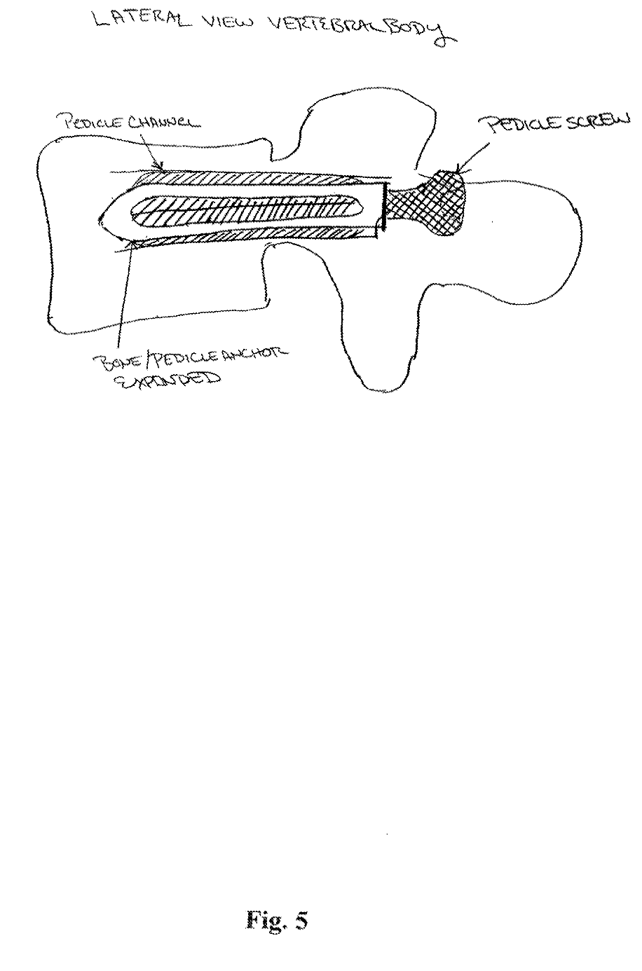

Referring to FIG. 5, a lateral view vertebral body is shown which depicts a pedicle channel, a pedicle screw, and an expanded bone/pedicle anchor.

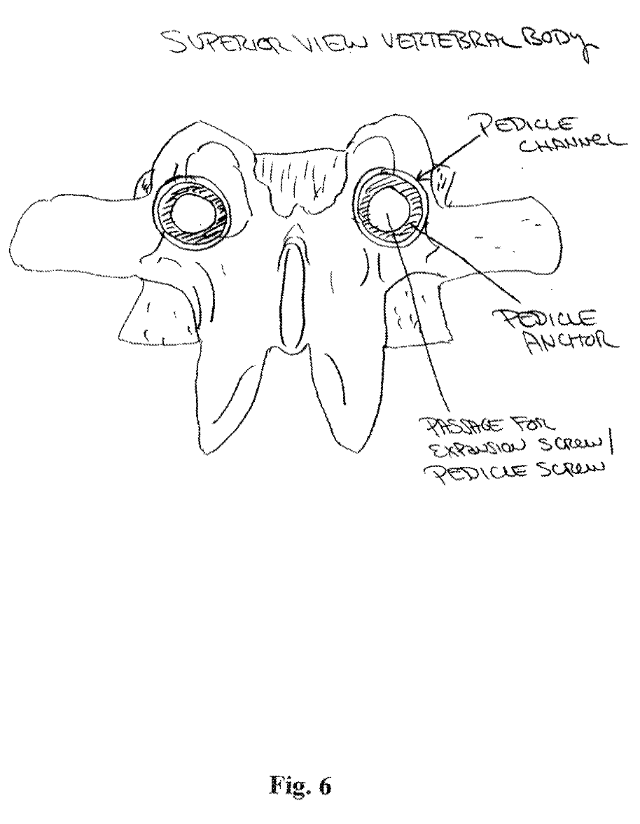

Referring to FIG. 6, a superior view vertebral body is shown which depicts a pedicle channel, a pedicle anchor, and a passage for expansion screw or pedicle screw.

While embodiments of the invention have been described above, it is evident that many alternatives, modifications and variations will be apparent to those skilled in the art. Accordingly, the embodiments of the invention, as set forth above, are intended to be illustrative, and should not be construed as limitations on the scope of the invention. Various changes may be made without departing from the spirit and scope of the invention. Accordingly, the scope of the present invention should be determined not by the embodiments illustrated above, but by the claims appended hereto and their legal equivalents.

I claim:

1. A bone anchor device, comprising:
a wall(s) that expand(s) within a pedicle channel or a long bone when a pedicle screw, a pedicle shaft or a cortical screw is inserted into the bone anchor device.

2. The bone anchor device of claim 1, wherein a portion of the bone anchor device extends beyond the pedicle into a vertebral body for added fixation within the vertebral body.

3. The bone anchor device of claim 1, wherein a portion of the bone anchor device extends beyond the far cortical wall when used in long bone fracture fixation.

4. The bone anchor device of claim 3, wherein an expansion tip prevents the bone anchor device from being withdrawn through the far cortex when expanded.

5. The bone anchor device of claim 1, wherein when used for pedicle fixation, the bone anchor device minimizes micro motion caused by fixation forces.

6. The bone anchor device of claim 5, wherein in the case of dynamic stabilization systems where a great deal of micro motion will be encountered at the screw-bone interface, the bone anchor device significantly decreases the effects of this motion on bone by acting as a transitional interface as well as motion that is absorbed by the bone anchor device itself.

7. The bone anchor device of claim 1 further utilized to provide safety to adjacent neural structures in the event a breach of the pedicle cortices is encountered.

8. The bone anchor device of claim 1, wherein the bone anchor device is comprised of allograft, PEEK, carbon fiber, or other biomaterial.

9. The bone anchor device of claim 1, wherein the walls are threaded externally to allow the bone anchor device to be threaded into a previously tapped pedicle or long bone.
